(19) United States
(12) Patent Application Publication
(10) Pub. No.: US 2003/0039708 A1
(43) Pub. Date: Feb. 27, 2003

(54) NON-MA HUANG HERB WEIGHT LOSS PRODUCT

(76) Inventor: Albert M. Fleischner, Westwood, NJ (US)

Correspondence Address: PHARMACEUTICAL PATENT ATTORNEYS, POHL & ASSOC. LLC, 55 MADISON AVENUE, 4TH FLOOR (P4014), MORRISTOWN, NJ, 07960-6397, US

(21) Appl. No.: 09/928,714

(22) Filed: Aug. 13, 2001

(51) Int. Cl. A61K 35/78; A61K 31/715

(52) U.S. Cl. 424/729; 424/773; 514/54

(57) ABSTRACT

Supplement compositions designed to support weight loss and increase energy while suppressing appetite.

| | | |
|---|---|---|
| Chromium (as chromium dinicotinate glycinate) | 25 mcg | to 200 mg |
| Vanadium (as vanadium amino acid chelate) | 25 mcg | to 100 mg |
| Glucomannan | 100 mg | to 500 mg |
| Green tea leaf extract (supplying 60 mg caffeine) | 50 mg | to 500 mg |
| Coleus forskohlii extract (10% forskolin) (tuber) | 50 mg | to 500 mg |
| Sodium carboxymethyl cellulose | 25 mg | to 250 mg |

| | |
|---|---|
| Excipients: | aa of each to produce a |
| Gelatin, Magnesium Stearate, Silica | suitable tablet |

### BACKGROUND

The prior art regarding this invention arises from distinct areas not heretofore combined to create new and useful formula sets or new and useful improvements thereof regarding a Solid-dosage Form of a Weight Loss Product.

This invention relates to the evolving science that a new and unique combination of coleus forskohlii extract (10% forskolin) (tuber), green tea extract (supplying 60 mg. caffeine), chromium (as chromium dinicotinate glycinate), vanadium (as vanadium amino acid chelate), and glucomannan, results in increased weight loss, increased energy and suppressed appetite without losing lean body mass.

Forskolin is an adenylyl cyclase activator that fully mimics thyroid-stimulating hormone and induces lipolysis, the breakdown of fat, in fat cells. The net result is the release of fatty acids from stored fat cells. Forskolin increases lean body mass while simultaneously reducing body fat and weight, thereby helping to optimize body composition.

When combined with the thermogenic green tea extract, glucomannan for satiety, chromium to reduce sugar cravings, and vanadium, an insulin mimic, the result is a weight loss product that is ephedra-free but still results in weight loss, increased energy, and appetite suppression.

Albert M. Fleischner, Ph.D., has a doctorate in Pharmaceutical Chemistry from Rutgers University and a degree in Pharmacy from Temple University. He has had over thirty years experience in the pharmaceutical industry with firms such as Schering Corporation, Roberts Pharmaceutical, Lehn & Fink Division of Sterling Drugs, Bradley Pharmaceutical Corporation, Amerchol Division of CPC and the Goen Group companies. He has a number of published papers and two previously granted patents and has several patents pending.

### SUMMARY

The invention discloses the formula sets that embody the invention of the supplement composition for increasing weight loss and energy levels while suppressing appetite. The formula increases fat loss, increases lean body mass, reduces weight, regulates insulin secretion, lowers systolic pressure, reduces appetite, and is very safe.

We now turn to discussing in great detail the best (or “preferred”) versions (or “embodiments”) of the invention.

A representative formula for Solid-Dosage Form of Weight Loss Product is as follows, one tablet contains:

| | | |
|---|---|---|
| Chromium (as chromium dinicotinate glycinate) | 150 | mcg |
| Vanadium (as vanadium amino acid chelate) | 50 | mcg |
| Glucomannan | 400 | mg |
| Green tea leaf extract (supplying 60 mg caffeine) | 300 | mg |
| Coleus forskohlii extract (10% forskolin) (tuber) | 250 | mg |
| Sodium carboxymethyl cellulose | 100 | mg |

| | |
|---|---|
| Excipients: | aa of each to make a |
| Gelatin, Magnesium Stearate, Silica | suitable tablet |

The scientific rationale for the formulation is as follows:

Forskolin is an extract of coleus forskohlii, a member of the mint family. The compound forskolin is known to enhance adenylate cyclase, an enzyme that splits a high energy molecule of adenosine triphosphate (ATP) to yield cyclic adenosine monophosphate (cAMP). AMP causes a chain of biochemical events that pace body metabolism and food-induced thermogenesis and provide mechanisms for controlling body composition and lean body mass.² The biochemical mechanism of maintaining or increasing lean body mass is related to the availability of cyclic AMP (cAMP).

By facilitating hormonal action, cAMP may regulate the body's thermogenic response to food, increase the body's basic metabolic rate, and increase utilization of body fat (since thermogenesis is preferentially fueled by fatty acids derived from body fat and/or food). These events also correspond to the buildup of lean body mass.³

Forskolin bypasses the adrenergic receptors which can often decrease with age and other physiological factors and has also been shown to counteract the decreased response of fat cells to epinephrine associated with aging. Normally, cAMP is formed when a stimulatory hormone (e.g. epinephrine) binds to a receptor site on the cell membrane and triggers the activation of adenylate cyclase.⁴ Since the mechanism of action does not involve non-selective binding to the adrenergic receptors, forskolin does not produce the unwanted side effects associated with their activations.⁵

Typically, an increase in cAMP leads to subsequent activation of protein kinase. Protein kinase has been shown to activate the enzyme lipase, which disposes of triglycerides, known as the building blocks of fatty tissue.⁶

A clinical study showed that 25 mg of forskolin twice a day can improve overall body weight by increasing lean body mass and by decreasing weight from body fat. Therefore, forskolin can potentially benefit not only overweight people but also those individuals who are active in training or body building and are looking to increase their lean body mass to body fat ratio.⁷ This is the key benefit of forskolin, an ability to improve body composition.

In a clinical study reported in the Journal of Biological Chemistry, forskolin was also found to fully mimic thyroid-stimulating hormone (TSH).⁸ TSH is a glycoprotein hormone secreted by the anterior lobe of the pituitary gland that stimulates and regulates activity of the thyroid gland, which is a two-lobed endocrine gland located in front of and on either side of the trachea that produces various hormones. The thyroid gland plays a major role in determining metabolic rate. An underactive thyroid can result in weight gain (or great difficulty losing weight), depression, fatigue, bloating and often a malaise and lack of energy, as well as cold extremities.⁹

Forskolin increases the heart rate with a positive inotropic action and lowers blood pressure.¹⁰ Forskolin also produces cAMP-independent effects associated with the modulation of membrane transport proteins. These include: adenylyl cyclase stimulation, glucose transport inhibitor, increased nicotinic receptor desensitization, and the inhibition of ligand- and voltage-gated ion channels.¹¹ It has been shown to inhibit platelet aggregation, further validating its beneficial cardiovascular effects.¹² Forskolin also benefits the management of allergic reactions^{13,14,} the maintenance of respiratory ^{15,16,} cardiac and circulatory health ^{17,18,} glaucoma¹⁹, ocular hypertension²⁰, psoriasis²¹, and is a safe and effective therapeutic approach in the management of vasculogenic impotence that is resistant to standard pharmacotherapy.²²

ForsLean™ is an extract of Coleus forskohlii root, standardized for 10 percent forskolin, used in our formula. In a study of overweight women given an amount of Forslean equivalent to that used in our formula for eight weeks, mean values for body weight and fat content significantly decreased, whereas lean body mass was significantly increased as compared to the baseline. A trend was observed of lower systolic/ diastolic pressure during the course of treatment.²³

Chromium is an essential nutrient required for sugar and fat metabolism. Normal dietary intake of chromium for humans is suboptimal. The estimated safe and adequate daily dietary intake for chromium is 50 to 200 mcg. Most diets contain less than 60% of the minimum suggested intake of 50 micrograms. Insufficient dietary intake of chromium leads to signs and symptoms that are similar to those observed for diabetes and cardiovascular disease. Supplemental chromium given to people with impaired glucose tolerance or diabetes leads to improved blood glucose, insulin, and lipid variables.²⁴

Chromium levels deplete with age²⁵ and illness²⁶, as well as with exercise.²⁷ Such depletion, when it occurs with excessive consumption of sugar and other carbohydrates, may result in glucose intolerance, glycosuria, hyperinsulinemia and hyperlipidemia.²⁸ The key benefit of chromium is in regulating and facilitating the efficient function of insulin. Chromium potentiates the action of insulin in vitro and in vivo.²⁹ As a cofactor in insulin utilization, chromium maintains proper blood sugar levels.³⁰

In a randomized, double-blind placebo-controlled clinical trial conducted at Wake Forest University, insulin resistance was reduced by a statistically significant 40% and this improvement was maintained at the end of eight months. The placebo group gained 6.5% abdominal fat while the chromium group gained just 1%,³¹

Chromium is an essential nutrient involved in the regulation of carbohydrate lipid metabolism and can break the cycle of overeating sweets. Eating sugar and high-fructose food and beverages causes chromium deficiency which can simultaneously cause drastic increases in insulin and glucose levels. This will trigger a person's craving for fattening sweets, which causes the cycle to repeat.³² When insulin is functioning efficiently, blood sugar and fatty acids metabolize properly, producing heat (thermogenesis) instead of weight gain.

In a second clinical study, chromium supplementation increased lean body mass in obese patients.³³ Dermatologists report that chromium appears to have value as an acne treatment.³⁴

Chromium dinicotinate glycinate is a polynicotinate. Polynicotinates have been shown to possess greater biological activity and are safer than other chromium supplements.^{35,36}

Vanadium compounds increase glucose transport activity and improve glucose metabolism.³⁷ It is also associated with greater insulin sensitivity.³⁸ Vanadium compounds mimic many of the metabolic actions of insulin both in vitro and in vivo and improve glycemic control in humans with diabetes mellitus. It also modulates insulin metabolic effects by enhancing insulin sensitivity and prolonging insulin action. The ability of vanadium compounds to ‘bypass’ defects in insulin action in diseases characterized by insulin resistance and their apparent preferential metabolic versus mitogenic signaling profile make them attractive as potential pharmacological agents.³⁹ Vanadium has demonstrated antihypertensive⁴⁰ and anorectic properties as well.⁴¹

Glucomannan is a dietary fiber derived from the tubers of amorphophallus konjac. When glucomannan is placed in water, it can swell up to 17 times its original volume.⁴² This is a greater volume than many other fiber supplements such as guar gum and pectin. In a double-blind study, participants taking glucomannan showed significant reductions in total cholesterol, LDL cholesterol and triglycerides. Systolic blood pressure was reduced with no significant increase in HDL cholesterol.⁴³ A second study showed similar results but also resulted in weight loss, whereas the placebo group gained weight over the length of the trial.44

A clinical study conducted at St. Michael's Hospital in Toronto concluded that adding glucomannan to conventional treatment may ameliorate glycemic control, blood lipid profile, and systolic blood pressure in high-risk diabetic individuals, possibly improving the effectiveness of conventional treatment in type 2 diabetes.⁴⁵ Yet another study found that glucomannan is very useful in the prevention and treatment of hyperglycemia.⁴⁶

In combination with diet, glucomannan was found to be more effective than diet alone for body weight, blood glucose, total serum cholesterol and hunger and satiety sensation.⁴⁷ In a second study, severely obese patients using diet plus glucomannan had a more significant weight loss in relation to the fatty mass alone, an overall improvement in lipid status and carbohydrate tolerance, and a greater adherence to the diet in the absence of any relevant side effects. Due to the marked ability to satiate patients and the positive metabolic effects, glucomannan was found to be particularly effective and well tolerated even in the long term treatment of severe obesity.⁴⁸ Glucomannan is also an ideal therapeutic tool in the management of chronic constipation symptoms.⁴⁹

Green tea (camellia sinensis) extract stimulates brown adipose tissue thermogenesis to an extent that is much greater than can be attributed to its caffeine content per se; its thermogenic properties reside primarily in an interaction between its high content in catechin-polyphenols and caffeine with sympathetically released noradrenaline (NA). Green tea extract is effective in stimulating thermogenesis by relieving inhibition at different control points along the NA-cAMP axis. Such synergistic interaction between catechin-polyphenols and caffeine to augment and prolong sympathetic stimulation of thermogenesis has value in assisting the management of obesity.⁵⁰

Epigallocatechin gallate from green tea polyphenols significantly reduced food intake, body weight, blood cholesterol and triglyceride, as well as growth of the prostate, uterus, and ovary; it may interact specifically with a component of a leptin-independent appetite control pathway.⁵¹ Green tea clearly has thermogenic properties, promotes fat oxidation⁵² and plays a role in the control of body composition via sympathetic activation of thermogenesis, fat oxidation or both.

In addition, the scavenging action of green tea extracts on singlet oxygen has a preventive effect on lipid peroxidation. Malondialdehyde production, a marker of lipid peroxidation, was found to be completely inhibited by a high concentration of green tea extract in the reaction system. In other words, green tea extract prevents unsaturated fats from being converted into saturated fats.⁵³

Green tea polyphenols have demonstrated significant antioxidant, anticarcinogenic, anti-inflammatory, thermogenic, probiotic, and antimicrobial properties in numerous human, animal, and in vitro studies. 54

This formula is intended to be offered as a Dietary Supplement under the Dietary Supplement Health and Education Act of 1994 (DSHEA) and is not intended to diagnose, treat, prevent, mitigate or cure any disease.

Without further elaboration, it is believed that one skilled in the art can, using the preceding description, utilize the present invention to its fullest extent. The specific formulas are included as a preferred embodiment of the composition formula ranges, and not to further qualify the description. Claim references to specific components include the component itself, as well as concentrates, metabolites, constituents, extracts or combinations of said ingredients.

### Footnotes

¹ http://www.samilabs.com/forslean/eephedrine.htm.

² Badmaev, V. et al. (2000) Forslean™: Ayurvedic herb Coleus forskohlii shows promise in enhancing lean body mass. Health Supplement Retailer. 7:30-32.

³ Ibid . . .

⁴ http://www.samilabs.com/forslean/pharmacologic.htm.

⁵ http://www.samilabs.com/forslean/ephedrine.htm.

⁶ Op cit, Badmaev, p.32.

⁷ Ibid.

⁸ Pomerance, M. et. al. (2000) Thyroid-stimulating hormone and cyclic AMP activate p38 mitogen-activated protein kinase cascade. Involvement of protein kinase A, Rac 1 and reactive oxygen species. J. Biol. Chem. 9/26.

⁹ http://www.ivillage.com/diet/experts/wlcoach/diet/articles/0,5050,91591,00.html.

¹⁰ Lindner, E. et. al. (1978) Positive inotropic and blood pressure lowering activity of a diterpene derivative isolated from Coleus forskohli: Forskolin. Arzneimittelforschung. 28(2):284-9.

¹¹ Laurenza, A. et al (1989) Forskolin: a specific stimulator of adenylyl cyclase or a diterpene with multiple sites of action? Trends Pharmacol Sci Nov;10(11):442-7.

¹² op cit. Lindner.

¹³ Kreutner, W. et al. (1985) Bronchodilator and antiallergy activity of forskolin. Eur J Pharmacol 111:1-8

¹⁴ Marone, G. et al. (1986) Forskolin inhibits release of histamine from human basophils and mast cells. Agents and Actions, 18(1/2):96-99.

¹⁵ Bauer, K. et al. (1993) Pharmacodynamic effects of inhaled dry powder formulations of fenoterol and colforsin in asthma. Clin. Pharmacol. Ther. 53:76-83.

¹⁶ Lichey, J. et al. (1984) Effect of forskolin on methacholine-induced bronchoconstriction in extrinsic asthmatics. The Lancet: July 21:167.

¹⁷ Op cit. Lindner.

¹⁸ Dubey, MP, et al.(1981) Hypotensive diterpene from Coleus forskohlii. J Ethnopharmacology 3:1-13.

¹⁹ Meyer, B H, et al. (1987) The effects of forskolin eye drops on intraocular pressure. S Afr Med J 71(9):570-571.

²⁰ Peng, T. et al. (1992) The experimental studies of the effect of forskolin on the lowering of intraocular pressure. Yen Ko Hseu Pao, 8(4):152-155.

²¹ Ammon, H P T and Muller (1989) Forskolin: from an Ayurvedic Remedy to a Modern Agent Planta Medica. Vol 51, 475-476.

²² Mulhall, J P et al. (1997) Intracavernosal forskolin: role in management of vasculogenic impotence resistant to standard 3-agent pharmacotherapy. J Urol. 158(5):1752-9.

²³ Op cit. Badmaev, p. 32.

²⁴ Anderson, R A (1997) Chromium as an essential nutrient for humans. Regul Toxicol Pharmacol Aug;26(1Pt2):S35-41.

²⁵ Dolby, Victoria (1998) Chromium at the Energy Crossroads. Better Nutrition Magazine May.

²⁶ Pekarek, R S et al (1975) Relationship between serum chromium concentrations and glucose utilization in normal and infected subjects. Diabetics Apr;24(4):350-3.

²⁷ Rubin, M A et al (1998) Acute and chronic resistive exercise increase urinary chromium excretion in men as measured with an enriched chromium stable isotope. J Nutr Jan;128(1):73-8.

²⁸ Boyle, E Jr. et al (1977) Chromium depletion in the pathogenesis of diabetes and atherosclerosis. South Med J Dec;70(12):1449-53.

²⁹ Mertz, W (1993) Chromium in human nutrition: a review. J Nutr Apr;123(4):626-33.

³⁰ Anderson, R A (2000) Chromium in the prevention and control of diabetes. Diabetes Metab Feb;26(1):22-7.

³¹ http://www.pslgroup.com/dg/2D74A.htm

³² Eades, M. D., Michael R. et al (1996) Protein Power (New York: Bantam Books), page 122.

³³ Anderson, R A (1998) Effects of chromium on body composition and weight loss. Nutr Rev Sep;56(9):266-70.

³⁴ McCarty, M (1984) High-chromium yeast for acne? Med Hypotheses Jul;14(3):307-10.

³⁵ Stearns, D M et al (1995) FASEB J Chromium picolinate produces chromosome damage in Chinese hamster ovary cells. Dec;9(15):1643-8.

³⁶ Olin K L, et al. (1994) Comparative retention/absorption of chromium from chromium chloride, chromium nicotinate and chromium picolinate in a rat model. American College of Nutrition 33^{rd} Annual Meeting University of California Berkeley-Davis.

³⁷ Barceloux, D G (1999) Vanadium. J Toxicol Clin Toxicol;37(2):265-78.

³⁸ Preuss, H G (1997) Effects of glucose/insulin perturbations on aging and chronic disorders of aging: the evidence. J Am Coll Nutr Oct;16(5):397-403.

³⁹ Fantus, I G et al. (1998) Multifunctional actions of vanadium compounds on insulin signaling pathways: evidence for preferential enhancement of metabolic versus mitogenic effects. Mol Cell Biochem May;182(1-2):109-19.

⁴⁰ Verma, S et al. (1998) Nutritional factors that can favorably influence the glucose/insulin system: vanadium. J Am Coll Nutr Feb;17(1):11-8.

⁴¹ Brichard, S and Henquin, J C (1995) The role of vanadium in the management of diabetes. Trends Pharmacol Sci Aug:16(8)265-70.

⁴² http:/www.tnp.com/substaiLce.asp?ID=1036

⁴ Arvill A, Bodin L (1995) Effect of short-term ingestion of konjac glucomannan on serum cholesterol in healthy men. Am J Clin Nutr 61:585-589.

⁴⁴ Reffo, G C et al (1990) Double-blind evaluation of glucomannan versus placebo in postinfarcted patients after cardiac rehabiliation. Curr Res Ther. 47:753-758.

⁴⁵ Vuksan, V et al (2000) Beneficial effects of viscous dietary fiber from konjac-mannan in subjects with the insulin resistance syndrome: results of a controlled metabolic trial.

Diabetes Care Jan;23(1):9-14.

⁴⁶ Huang, C Y et al (1990) Effect of konjac food on blood glucose level in patients with diabetes. Biomed Environ Sci Jun;3(2):123-31.

⁴⁷ Cairella M, Marchini G (1995) Evaluation of the action of glucomannan on metabolic parameters and on the sensation of satiation in overweight and obese patients. Clin Ter Apr;146(4):269-74.

⁴⁸ Vita, P M et al (1992) Chronic use of glucomannan in the dietary treatment of severe obesity. Minerva Med Mar;83(3):135-9.

⁴⁹ Marzio, L et al (1989) Mouth-to-cecum transit time in patients affected by chronic constipation: effect of glucomannan. Am J Gastroenterol Aug;84(8):888-91.

⁵⁰ Dulloo, A G et al (2000) Green tea and thermogenesis: interactions between catechin-polyphenols, caffeine and sympathetic activity. Int J Obes Relat Me tab Disord Feb;24(2):252-8.

⁵¹ Kao, Y H et al (2000) Modulation of endocrine systems and food intake by green tea epigallocatechin gallate. Endocrinology Mar;141(3):980-7.

⁵² Dulloo, A G (1999) Efficacy of a green tea extract rich in catechin polyphenols and caffeine in increasing 24-h energy expenditure and fat oxidation in humans. Am J Clin Nutr Dec;70(6):1040-5.

⁵³ Wu, Y (1993) Scavenging action of green tea extracts on singlet oxygen and preventive effect on lipid peroxidation.Chung Kuo I Hsueh Ko Hsueh Yuan Hsueh Pao Oct;15(5):354-9.

⁵⁴ Ed. (2000) Green tea. Altern Med RevAug;5(4):372-5.

I claim:

1. A composition of matter intended to support weight loss,
(a) said composition of matter containing (i) Chromium and (ii) Vanadium, and (iii) Glucomannan, (iv) Green tea leaf extract, and (v) Coleus forskohlii extract, and (vi) sodium carboxymethyl cellulose.
(b) said composition of matter intended for ingestion in capsule, tablet or liquid form; and
(c) said composition of matter not represented for use as a conventional food or as the sole item of a meal or diet; and
(d) said composition of matter labeled as a supplement for use in or by humans.

2. A composition of matter intended to support weight loss.
(a) said combination of matter containing (i) Coleus forskohlii extract and (ii) glucomannan, and (iii) additional dietary substances which support the primary ingredients' activities.
(b) same
(c) same
The supplement of claim wherein said ingredients are present in any combination thereof in the following approximate amounts:

| | | |
|---|---|---|
| Chromium (as chromium dinicotinate glycinate) | 150 | mcg |
| Vanadium (as vanadium amino acid chelate) | 50 | mcg |
| Glucomannan | 400 | mg |
| Green tea leaf extract (supplying 60 mg caffeine) | 300 | mg |
| Coleus forskohlii extract (10% forskolin) (tuber) | 250 | mg |
| Sodium carboxymethyl cellulose | 100 | mg |
| | |
|---|---|
| Excipients: | aa of each to produce a |
| Gelatin, Magnesium Stearate, Silica | suitable tablet |
